(19) United States
(12) Patent Application Publication
(10) Pub. No.: US 2014/0196792 A1
(43) Pub. Date: Jul. 17, 2014

(54) Three-Way Valve Case Apparatus

(71) Applicant: Global Medical Innovations, LLC, Woburn, MA (US)

(72) Inventor: Mario Torres-Leon, Woburn, MA (US)

(73) Assignee: Global Medical Innovations, LLC, Woburn, MA (US)

(21) Appl. No.: 14/158,460

(22) Filed: Jan. 17, 2014

Related U.S. Application Data
(60) Provisional application No. 61/753,623, filed on Jan. 17, 2013.

(51) Int. Cl. F16K 27/12

(52) U.S. Cl. CPC F16K 27/12
USPC 137/1; 137/377

(57) ABSTRACT

The present invention relates to a three-way valve case that has a top member, bottom member, closure member, three openings, and a hinge allowing the case to be opened and closed. The case may be adapted to receive a three-way valve. Apart from a case adapted to receive a three-way valve, a three-way valve case apparatus is also disclosed. The apparatus includes a three-way valve having three ports, a base, and an operator; and a case having a top member, bottom member, hinge, openings to receive the ports of the valve, and a closure member to support the closed state of the case.

### RELATED APPLICATIONS

This application claims the benefit of U.S. Provisional Application No. 61/753,623, filed Jan. 17, 2013.

The entire teachings of the above application are incorporated herein by reference.

### BACKGROUND OF THE INVENTION

Millions of patients around the world need placement of drainage catheters each year due to a variety of medical conditions. These catheters are placed either via traditional surgical techniques or via image-guided minimally invasive radiological procedures. As an alternative to an uncontrolled connection to a drainage bag, or as an alternative to the usage of a drainage bag, it is possible to use a valve to control the flow of fluids from the patient. Such a control, in a majority of cases, is accomplished by connecting a drainage catheter to a drainage bag via a three-way valve. The three-way valve, in addition to allowing a control of the flow from the patient to the drainage bag, also allows flushing of the catheter and aspiration of fluid from the patient.

Even though using three-way valves provides some advantages over not using them, patients often report discomfort and frustration due to the usage of such valves, which creates a dilemma with two unappealing options.

### SUMMARY OF THE INVENTION

It has been observed that when patients use three-way valves, they often face tremendous discomfort when lying on the valves. Additional observations showed frequent skin breakdown in areas where the patient's skin is exposed to a valve. Further incidents indicated that the way these valves are used has been allowing inadvertent changes in the state of the ports of the valve.

Building on these problems, a three-way valve case apparatus has been developed and is the focus of the present invention. The apparatus of the present invention reduces discomfort due to the sharp features of three-way valves, reduces skin irritation due to the rough surface of three-way valves, and reduces incidences of inadvertent changes between open and closed states of the ports.

In particular, the present invention, in an embodiment, is a medical three-way valve and a case. The phrase “three-way valve,” for brevity also referred to as a “valve,” has three ports: a first port, a second port, and a third port. Together, the three ports are a part of the housing of the valve. In an embodiment, the valve can be used to stop the flow of fluid drained from the patient, or direct the fluid such that the fluid enters one port of the valve and exits another port, as desired. In another embodiment, fluid communication occurs among all the ports. The fluid communication between ports is controlled via a valve operator. The valve operator has a mechanism that allows fluid communication to occur between at least two of the ports, when the operator is in use. When the operator is not in use, fluid communication can be blocked between any two ports. The other component of the inventive apparatus, the case, goes around the valve and has two members connected with a hinge. One of the two members is referred to as the top member and the other one as the bottom member. This naming is for convenience only and does not limit the relative positions of the members with respect to the ground. Using the hinge allows the user to articulate the two members between and at open and closed positions of the case. The case is opened typically to make changes to the valve, or sometimes to check the status of the valve. To be able to receive the valve, the case has openings to receive the three ports of the valve. To accommodate the valve, the case has three openings to receive the three ports of the valve. These three openings are referred to as the first opening, the second opening, and the third opening. For convenience of description, the first opening is intended for the first port, the second opening for the second port, and the third opening for the third port. Because the external surface of the case need not be a constant surface, due to changing between open and closed states, the three openings of the case are defined functionally. Namely, the case is to have a set of openings to receive the ports of the valve, but the precise numbering of such openings can be different as long as the function of the openings is achieved. For example, some embodiments have a case that has three openings in its closed position; however, the same three openings appear as six partial openings in its open position. A more particular example is a case having three disk shaped openings in its closed state that appear as six half-disk shaped openings in the open form of the case. The embodiments in which different openings are connected with channels or extended openings, literally creating unified openings and thus reducing the number of total openings from three to fewer than three, are also encompassed within the present invention. The embodiments of the present invention also include a closure member that can keep the top and bottom members of the case in the closed position. The closed position, as used here, refers to the closed position of the case as a whole and includes the set of states of the case that shield the valve from the user. When some embodiments of the present invention are in use, the closed position of the case makes the valve operator inaccessible to the user and the valve out of touch of the user's skin. Additional embodiments have different or more specific features. For example, some embodiments have a snap closure, and some embodiments have a flexible hinge, such as a living hinge. In other embodiments, some of the openings are formed by only portions of either the top or the bottom member, or by portions of both the top and the bottom member. Alternative embodiments have a bottom member with a smooth outer surface. Some of these embodiments can be made from plastic materials, as well as from polymers having different degrees of thermosetting or thermoplastic properties. Some examples of thermoplastic polymers that can be used are polyethylene (e.g., high density polyethylene), polypropylene, polystyrene, polyvinyl chloride, polytetrafluoroethylene, and combinations of the foregoing. The material of the case, in an embodiment, is an organic polymer. Additional embodiments have a case that has a surface that is smother than the surface of the valve that it covers. Other embodiments of the present invention include a handle as part of the valve. Such a handle is in communication with the valve operator, and thus lets the user change the state of the valve ports. The case of the apparatus, in some embodiments is able to cover the valve and the valve handle. In embodiments that have a handle that can be connected and disconnected, the case need not cover the handle. To enable the user to see the state, whether they are open or closed, of the ports, some embodiments have a case that has a transparent or translucent portion. In some embodiments, the case has multiple transparent or translucent portions; in others it is entirely transparent or translucent.

The present invention also includes an embodiment that is a case for covering a medical three-way valve. The “three-way medical valves,” for brevity also referred to as “valves,” that the case is intended to cover are composed of a valve housing, a valve operator, and a valve handle. The housing of the three-way valves includes a valve base as well as three ports: a first port, a second port, and a third port. The valve operator has a mechanism that allows fluid communication between two of the ports to occur. Alternative mechanisms of the valve operator allow fluid communication to be blocked, or to occur between more than two ports. The valve handle communicates with the valve operator, and allows the user to control the operation of the valve. A case embodied by the present invention includes two members, one designated as the top member, and the other designated as the bottom member. These two members are connected by a hinge that allows the two members to be articulated at or between an open position and a closed position. A closed position refers to a position of the case that shields the valve from the user, for example by making the handle inaccessible or by keeping the surface of the valve out of touch of the user, and an open position refers to a position of the valve that allows the user to access the valve handle. The three ports of the valve, namely the first port, the second port, and the third port are received by three openings of the case, namely the first opening, the second opening, and the third opening, respectively. The designations first through third for the openings are defined with the first through third ports of the valve as a reference; as such, as long as they receive the respective ports, they do not need to be entirely isolated openings, separate openings, or integral openings. In some embodiments, the openings are partial openings in the open state of the case. Some embodiments of the present invention also include a closure member that can maintain the top and the bottom members in the closed position, e.g., maintain the case in a closed position by accordingly aligning the top and the bottom members. Some of these embodiments make the valve operator and the valve handle inaccessible to the user such that when the case is in a closed position and the case is in use, the parts of the valve that are inside of the case do not come into contact with the skin or clothes of the user. In alternative embodiments, the case has dimensions that prevent the valve from sliding inside the case; thus, essentially keeping the valve in place. In other embodiments, the closed position of the case has its width, length, and height dimensions in the ranges between about 1.5″ and about 2.0″, about 1.5″ and about 2.0″, and about 0.75″ and about 1.2″, respectively. Particular embodiments of the case have a bottom member that has an inner surface that is adapted to receive the valve base. This is accomplished by, for example, the inner surface of the bottom member having a fastener that interacts with the base of the valve. In an embodiment, a specially designed valve or a currently existing valve can be placed onto such a fastener. The fastener can be, e.g., a snap, a hook and loop arrangement, or any other structure that creates a fit between the valve and the inner surface of the bottom member stronger than a fit between the valve and the inner surface of the bottom member without such a fastener. In a particular example for creating such an adaptation between the inner surface of the bottom member and the valve is an embodiment that has an adhesive member that accomplishes the attachment between the inner surface of the bottom member and the valve. The adhesive member, in an embodiment, is adhesive on only one side; in others, on more than one side. The adhesive member, in some embodiments, is removable from the case; in others, it is integrally attached to the case. Additional embodiments have a bottom member that can receive a valve in a removable way, e.g., valves can be attached, later removed, and a new one or the same one reattached.

The present invention also embodies kits that have a three-way vale and a case that receives the three-way valve. The included three-way valve has a housing and a valve operator. The included housing has three ports: a first port, a second port, and a third port. The included valve operator has a mechanism that allows fluid communication between at least two ports. In different embodiments, the fluids can flow from one port to another, fluids can flow from one port to two others, and/or fluids can flow from two ports into one. The mechanism of the valve operator also allows the fluid flow to be blocked. The other part of the kit, the case, receives the three-way valve. The case has a top member, a bottom member, a hinge, three openings, and a closure member. The hinge connects the top and the bottom members to each other, and allows their relative positions to be changed so that the case can be opened or closed. Of the three openings of the case, the first opening receives the first port, the second opening receives the second port, and the third opening receives the third port of the valve. These three openings, in separate embodiments, can be distinct or not, part of only one member or not, and indivisible with respect to the open versus closed states of the case or not. The included closure member helps maintain the top member and the bottom member in the closed position, in other words, the closure member facilitates keeping the case in a state that shields the valve from the user by locking in the relative positions of the top and bottom members. Some of the embodiments make the valve operator inaccessible to the user and prevent substantial contacts between the user and the three-way valve when the case is used in a closed position.

Methods of using a case of a three-way valve are also encompassed by the present invention. The case is to be used for three-way valves that have a housing and a valve operator. The housing of the three-way valve has a first port, a second port, and a third port. The valve operator has a mechanism that allows fluid communication to happen between at least two ports when the mechanism of the valve operator is in use. By the valve operator mechanism being in use, it is referred to the states of the ports in which not all of the ports are blocked. Blocking of all the ports is one of the uses of the valve operator and the valve; thus, such a use and method is also included among the embodiments of the present invention. However, as described herein, when the valve operator mechanism is in use, fluid communication happens between at least two of the ports, for example between two ports or between three ports. The methods of the present invention also embody situations in which the three-way valve is attached to a catheter being used by an individual. The case being used in the methods disclosed receives a three-way valve. The case is formed by a top member, a bottom member, there openings, a hinge, and a closure member. Among these three openings, the first opening receives the first port, the second opening receives the second port, and the third opening receives the third port. The hinge allows the relative positions of the top member to the bottom member to be changed so that the case can be switched between open and closed positions. The closure member enables the user to close the case and to keep the case in a closed position. Among the methods embodied by the present invention is the method of placing a three-way valve in a case and then engaging the top and the bottom members so that the case is put into a closed position. As a result of such a method, the valve operator becomes inaccessible to the user, and the three-way valve becomes shielded from the user. Alternative embodiments also include the steps of engaging the top and bottom members so that the case is put into an open position and then engaging the handle that communicates with the valve operator. In some embodiments, these method steps can be performed by an individual, the specific user of the device, or by a health care professional. Additional embodiments include the step or steps of connecting tubing from the first port, the second port, the third port, or any combination thereof. Further embodiments include the step or steps of disconnecting tubing from the first port, the second port, the third port, or any combination thereof Also included are methods that include the step of aspirating a fluid through a port using the three-way valve. The methods of flushing through a port of the three-way valve are included among the embodiments of the present invention as well.

A similar set of methods to the methods of using a case of a three-way valve are included, in which the used apparatus is a case together with the three-way valve. The case and three-way valve, in other words the three-way valve case apparatus, can be any of the embodiments disclosed as part of the present invention. The methods of using such an apparatus include steps of placing the ports of the three-way valve in a desired position and engaging the top and bottom members of the case to put the case in a closed position. Such methods make the valve operator inaccessible to the user and shield the three-way valve from the user. Additional methods include the steps of engaging the top and the bottom members to put the case in an open position and then engaging the handle of the valve as desired. Such steps, in some embodiments, are performed by an individual or by a specific health care professional. Other encompassed methods include the steps of connecting tubing from any single or multiple ports, among the first, second, and the third port, and either separately or in combination, the steps of disconnecting tubing from any single or multiple ports among the same set. Also included are methods of aspirating fluid through a port of the three-way valve as well as methods of flushing through a port of the three-way valve.

The embodiments of the present invention have numerous advantages. For example, being able to change three-way valves without needing an additional case keeps costs down. Being able to use a variety of third-party three-way valves enables the case to be versatile. Using a three-way valve encased in a case makes the exterior of the valve harmless to the user, as the user's skin is shielded from the exterior of the valve. This prevents abrasion or other types of damage from the valve occurring on the user's skin. The smoother surface of the case reduces any amount of discomfort that the patient may have experienced. In addition, the valve being guarded from the user also ensures that the handle of the valve is guarded from the user; thus, the probability of the states of the ports inadvertently being changed is significantly reduced.

### BRIEF DESCRIPTION OF THE DRAWINGS

The foregoing and other objects, features, and advantages of the invention will be apparent from the following more particular description of preferred embodiments of the invention, as illustrated in the accompanying drawings in which parts are referred to by reference characters across different views. The drawings are not necessarily to scale, emphasis instead being placed on illustrating the principles of the invention.

FIG. 1A is a schematic of a frontal view of an embodiment of an apparatus that has a case and a three-way valve. Shown are a top member, bottom member, hinge, first opening, second opening, third opening, first port, second port, third port, handle, and closure members. The case in the drawing is in an open position.

FIG. 1B is a schematic of a side view of an embodiment of an apparatus that has a case and a three-way valve, seen from a side that has a port connecting to a drainage bag, in which the case is in an open position.

FIG. 1C is a schematic of another side view of an embodiment of an apparatus that has a case and a three-way valve that has a port for flushing. The case is in an open position.

FIG. 1D is a schematic showing a perspective view of an apparatus that has a case and a three-way valve. The drawing illustrates a top member, bottom member, hinge, first opening, second opening, third opening, valve housing, valve base, first port, second port, third port, and a handle.

FIG. 2A is a schematic showing a perspective view of an apparatus that has a case and a three-way valve, in which the case is in an open position.

FIG. 2B is a schematic showing front view of an apparatus that has a case and a three-way valve, in which the case is in a closed position and the three-way valve inside the case can be discerned due to the case being translucent.

FIG. 3A is a schematic showing a perspective view of a case, which has not yet received a three-way valve, in an open position. Among the features shown are the bottom inside surface of the case and the adhesive member of the bottom member.

FIG. 3B is a schematic showing a top view of a case, which has received another embodiment of a three-way valve, in an open position.

FIG. 4A is a schematic of a frontal view of a case of the present invention in an open position. Among the features shown are a top member, bottom member, hinge, an adhesive member, and closure members.

FIG. 4B is a schematic showing a side view of a case in an open position. The drawing shows the case having ports connectable to drainage bags. Among the visible features is the first opening. The first opening is shown as separated into two halves, as the case is in an open position.

FIG. 4C is a schematic showing a side view of a case in an open position. The case is seen from a side that allows flushing. Only a bottom member is visible, due to the other member being hidden behind the bottom member from the viewer's perspective.

FIG. 4D is schematic showing a perspective view of a case. Shown are a top member, bottom member, hinge, first opening, second opening, third opening, and the inside surface of the bottom member.

### DETAILED DESCRIPTION OF THE INVENTION

A description of preferred embodiments of the invention follows.

The present invention relates to a case for a three-way valve that can be used by individuals who are using three-way valves as part of a procedure that has biological fluids being drained from their bodies. The present invention also pertains to a three-way valve case apparatus. The embodiments of the present invention allow individuals to use three-way valves in a more comfortable and efficient manner.

Medical conditions in which the case or the apparatus of the present invention can be used include those in which biological fluids are drained. Examples of conditions that have bodily fluid drainage include abscesses at various locations including abdominal abscesses, kidney conditions that require percutaneous nephrostomy, non-kidney conditions that require percutaneous nephrostomy, bladder conditions due to neurogenic causes (e.g., spinal cord injury, spina bifida, and multiple sclerosis), bladder problems due to non-neurogenic causes (e.g., infravesical obstruction due to prostate enlargement, urethral strictures and post-operative urinary retention), sedation or anesthetization prior to medical operations, being comatose, not being able to use a toilet due to physical injury or paralysis, kidney diseases that need monitoring of urine output, complications due to C-sections, needs arising during the induction of labor (childbirth), prostatic hypertrophy, congenital defects or traumatic disruption of the urinary tract, obstructions such as kidney stones, cancer, conditions requiring urostomy, pyonephrosis, hydronephrosis, infections of the urinary bladder, damages to the urethra, urinary retention, urinary incontinence, problems arising out of surgeries, and other health problems.

Such conditions require the use of catheters, which are tubes that can be inserted into a part of the body. Catheters can be used to perform a range of different functions, including drainage of biological fluids from the body. They can be inserted through the skin (percutaneous), into a body cavity, into a duct (such as the urethra), or into a body vessel (such as the arteries, veins, and the capillaries). After insertion, they can be left in for a brief time such as during an operation, for a prolonged time such as a full day, or even permanently. They can also be used intermittently. An example of a catheter is the Foley catheter, which is used to drain fluids from the urinary bladder into an outside drainage bag.

In an embodiment, a three-way valve can be used in conjunction with a catheter. A three-way valve allows the user to turn on, turn off, divert, or adjust the flow from the catheter. A three-way valve can additionally allow aspiration of fluids and flushing of the catheter. Some individuals use three-way valves together with drainage bags, while some others use three-way valves without drainage bags being attached. The handle of a valve, when not used with the case of present invention, may accidentally or inadvertently be engaged by the patient. A patient, when not using the apparatus of the present invention, may mistakenly create undesired fluid communication between the ports of these valves when the valve configuration changes without the patient desiring it or even noticing it. Additionally, when not used with the present invention, the non-flat surface features of these valves cause discomfort to individuals, as these features can cause significant pain when the valves are close to the individuals' skin. The embodiments of the present invention ameliorate these problems.

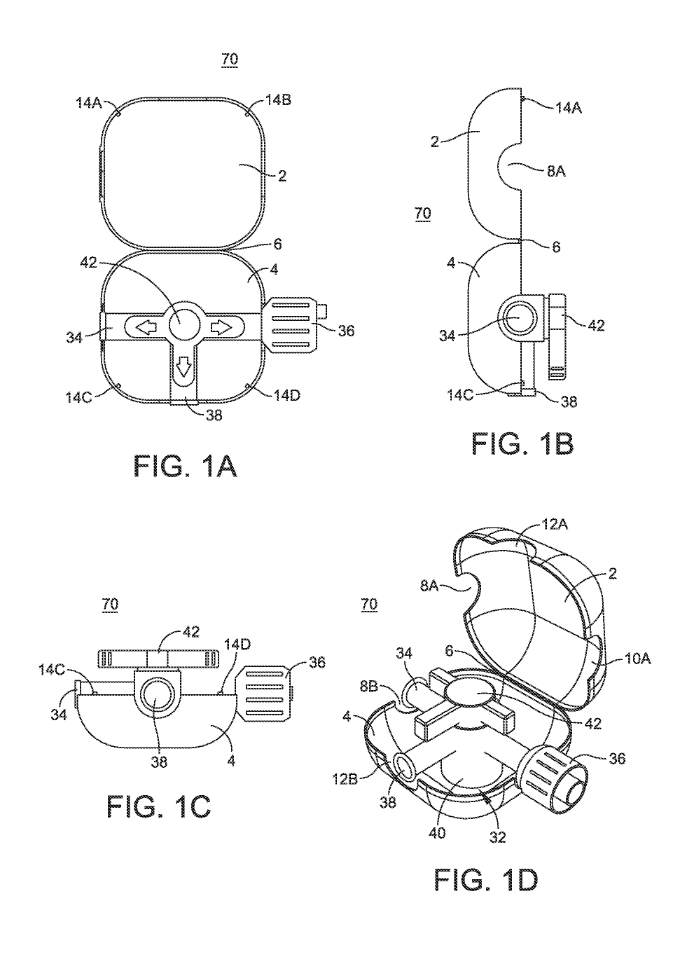

Referring to FIG. 1A, apparatus 70, also referred to as the three-way valve case apparatus 70, allows an individual to benefit from the advantages of using a three-way valve while facing reduced frustration due to the disadvantages of these valves. Individuals can use an embodiment of three-way valve case apparatus to connect it to catheters, to drainage bags, to flushing or aspirating units, or to a combination and the like thereof. As shown in FIG. 1A, apparatus 70 includes a case that has top member 2, bottom member 4, hinge 6, and closure members 14A, 14B, 14C, and 14D.

The top member and the bottom member together form a case that has or is able to receive a three-way valve. These two members, in different embodiments, are equivalent in size or not, and are similar in shape or not. In some embodiments, these parts of the case accommodate or receive a three-way valve and allow an individual to change the position of the handle. In alternative embodiments, the top or the bottom member, or both are caps. By a cap, it is referred to structures that can function as a cap, lid, top, dome, hood, roof, capsule, case, casing, covering, housing, jacket, sheath, shell, or the likes, multiples, or combinations thereof. The top and the bottom members fit snugly to each other around their edges, or in some embodiments, they leave some space at some parts of their edges between each other even after being closed. The bottom member is the one that typically receives the three-way valve; however, in some embodiments, for example in which the two halves are not seen as separable by a plane, the top member is the one to physically support the valve.

With the case being in use, it is referred to the case having a valve, and an individual being able to use the case and the valve at least temporarily without further adjustments to the position of the case.

The hinge is a bearing that allows changing the relative positions of the top member and the bottom member. In some embodiments, it is a flexible bearing and is composed of a separate substance than the substance of the top and the bottom members; for example, if the two members are made out of plastic, the hinge is made out of a flexible substance such as a cloth or leather. In other embodiments, the hinge, as a flexible bearing, is composed of the same material as that of the top and bottom members, or a part of at least one of the top or the bottom member. A particular example for the hinge is a living hinge, which would be made from the same material as at least one of the two members of the case. For example, in some embodiments, the living hinge is made out of plastics such as polypropylene or polyethylene. The section to serve as the living hinge, in alternative embodiments is made by various methods including designing the molding process to make the hinge area thinner, by scoring, by coining, or by a combination thereof. Techniques and improvements known in the art such as orienting the fibers of the plastic across the hinge to make the living hinge more durable, or other techniques and improvements later developed can be used in other embodiments. In some embodiments, the hinge is simply a fold line between the top and the bottom members. In alternative embodiments, the hinge is a bearing other than a flexible bearing, and is made of separate moving parts. Some examples for the hinge include a barrel hinge, mortise hinge, pivot hinge, or the like thereof. In some embodiments, the term hinge as used herein refers simply to the area on one side, for example the back side, of the case. In such as case, it is possible to separate the top and bottom members of the case and to combine them back into a stable close state. Such a mechanism is accomplished for example by a structure for a hinge that allows snug fitting of the top and bottom members to each other, whose separation would require application of pressure.

With the hinge being in use, it is referred to the positions in which there is a bearing of force by the hinge. For example, open positions cause some force to be exerted onto the hinge, since the hinge keeps the top and the bottom member in place. Movements between states likewise cause some force to be exerted onto the hinge, since the hinge allows the articulation between these states. In these definitions, distinction is not made between the directionality of the force, e.g., whether it is onto the hinge or from the hinge.

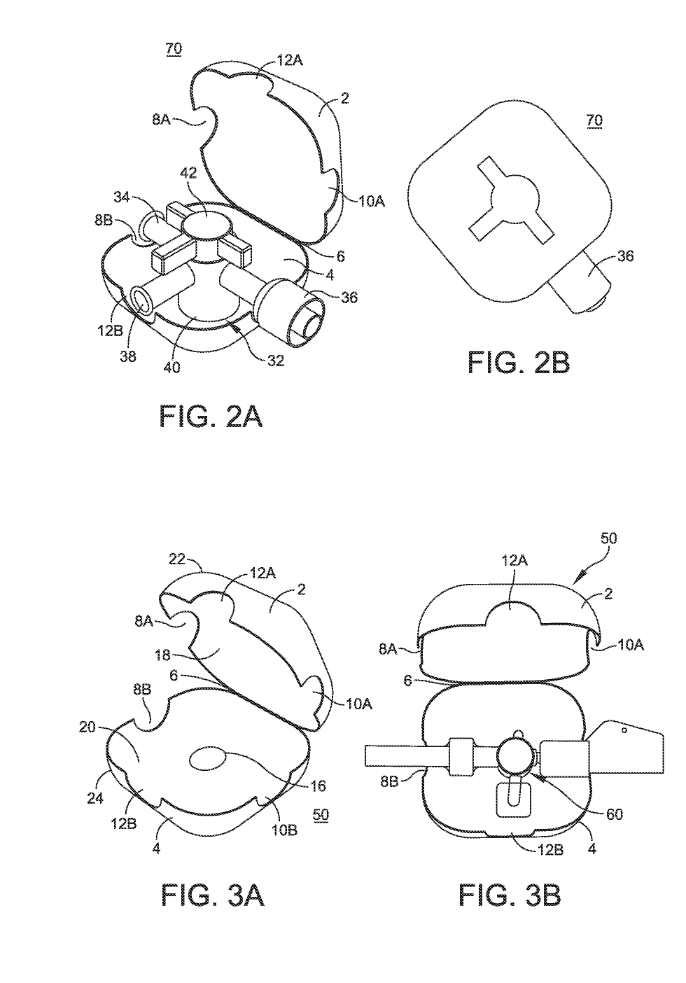

The case, in alternative embodiments, has a range of positions, including closed and open positions. In some embodiments, a hinge allows transitioning between these various positions. The hinge keeps a part of the top and the bottom members relatively close to each other, whereas allowing the parts of the two members that are distal to the hinge to move away from or toward each other, in a sense by being articulated between a closed and an open state. By this articulation, it is meant that the case can be placed in an open position, a closed position, or in positions that are intermediates between the two. FIG. 2A shows the case in an open position, whereas FIG. 2B shows the case in a closed position.

In some embodiments, the closure member is any fastener that enables the closed case to remain in the closed state. An example for a closure member is a snap. Other examples include a fastener, tie, securing means, strap, series of snaps, button, series of buttons, hook, series of hooks, elastic member, position adjuster, key-lock arrangement, lace, clip, series of clips, zipper, metal loop, roller loop, drawstring-and-cord lock arrangement, hook-to-hook arrangement, hook-and-loop arrangement, bungee cord-and-cord lock arrangement, block and tackle arrangement, rope and hook arrangement, chord and hook arrangement, snap hook, trap-lock, plastic trap-lock, magnet, slide, plastic slide, adhesive, as well as the likes, multiples, or combinations thereof. Fasteners known or developed in the future can be used so long as they allow opening and closing of the case. These fasteners can be obtained or purchased through commercially available means, for example from 3M Corporation (St. Paul, Minn.), from National Webbing Products Co. (Plainview, N.Y.), or from National Molding Corporation (Miami Lakes, Fla.).

Also shown in FIG. 1A, Apparatus 70 includes a three-way valve that has first port 34, second port 36, third port 38, and handle 42.

In some embodiments, the first port is used to connect to a drainage bag, the second port to connect to a catheter, and the third port to connect to flushing or aspirating units. In alternative embodiments, drainage bags are not used, as some individuals use valves instead of bags, as opposed to in addition to bags. This makes their condition easier to manage, and makes it easier to engage in social activities, since valves are more discreet than bags. Some individuals drain through their valves periodically or as needed. This helps the bladder to increase its capacity; as otherwise, the bladder can lose its tone and regular ability to hold urine. In embodiments that do not make use of a drainage bag, the first port is connected to an elongating tube, is not connected to any tube, or is connected to a temporary collection unit.

The three-way valve is a valve that has three ports, and allows or disallows flow of a fluid between at least some of the ports. In addition to the ports, the three-way valve has a base, a handle, and an operator. The valve, in some embodiments is an integral part of the catheter; in others, it is a separate item that connects to a catheter. Typically, the valve allows the user to block the regular fluid flow when drainage is not needed, and allows the user to permit fluid flow when drainage is desired. In additional embodiments, the flow is directed through alternative inlet and outlet ports, and/or a port or ports allow flushing or aspiration of the valve, catheter, or a part of the patient. Three-way valves are typically made from materials like polypropylene, polyvinyl chloride, and rubber. Placement and removal of valves requires usage of aseptic techniques, to reduce the probability of any infection. The valves typically allow for a flow of about 290-325 mL/min, and typically are changed every 5-7 days. Some valves can be purchased from manufacturers such as Braun Medical, Inc. (Bethlehem, Pa.), C. R. Bard, Inc. (Murray Hill, N.J.), Coloplast (Minneapolis, Minn.), Merit Medical Systems, Inc. (South Jordan, Utah), and Flexicare (Mountain Ash, UK).

In an embodiment, the valve is prevented from sliding within the case. By sliding, it is referred to the valve not being able to move more than a certain distance in a direction during a period. For example, a valve is referred to as being prevented from sliding if it moves less than 1%, 5%, 10%, or 20% during a period of 1 hr, 2 hrs, 4 hrs, 6 hrs, or 24 hrs. In one embodiment, a valve can be said to be prevented from sliding if it moves less than 5% (e.g., the valve handle rotates by less than 18 degrees) during a period of 24 hours, or, in another embodiment, if it moves less than 1% (e.g., the valve handle rotates by less than 3.6 degrees) during a period of 6 hours.

One way in which the valve is prevented from sliding is accomplished is simply through avoiding external objects, including part of the user, from coming into contact with the handle. Another way, in some embodiments, is through a tight fit between the valve and the case surrounding it. In one embodiment, the top member of the case is manufactured so as to have grooves that accommodate the three bars of the handle (e.g., T-shaped groove pattern), so that conformations of the handle that change from one set of open/closed ports to another set are forbidden while the case is in a closed position. Other types of indentations of the inner surface of the top member, whether complementary in shape to the handle or not, are also encompassed by the disclosed invention.

The ports of a three-way valve, as desired, can be in an open, closed, or partially open state. Depending on the mechanism of the operator, different embodiments allow maximal or minimal flow rate by adjusting the handle between discrete positions. Again, depending on the mechanism of the operator, alternative embodiments attain states in which the flow is adjusted from minimal to maximal with a plurality of degrees of flow in between. Such a degree of flow, in some embodiments is attained by regulating or metering types of valve fittings.

The handle allows the user to control the operator, and thus the state of the ports. In some embodiments, it is automatic e.g., based on pressure. In other embodiments, it is mechanical, e.g., T-shaped or L-shaped. Some embodiments include a ball element (or butterfly), globe element, gate element, needle element, or shuttle element. Alternative embodiments allow a quarter turn to activate the full range of available positions. Other embodiments allow lesser or more turns to reach the full range of positions. Other handles known in the art or developed in the future can be substituted as well.

The state of the valve operator being in use, or the state of the mechanism of the valve operator being in use refers to the state in which the flow of fluids between at least two of the ports is allowed. Conversely, the valve operator, or the mechanism of the valve operator not being in use refers to the state of fluid communication between ports being blocked.

Fluid communication refers to the fluid being able to flow between two or more ports. Some examples of fluid communication include the flow of drainage fluids from second port to the first port, from second port to the third port, from second port to both the first and the third port, from third port to the first port, from third port to the second port, and from third port to both the first and the second ports.

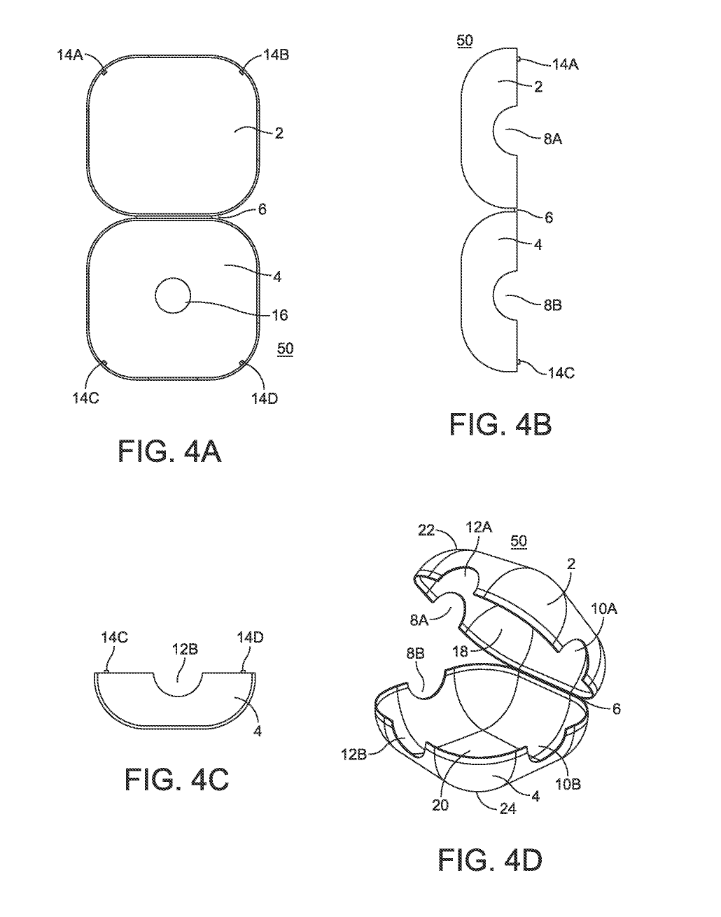

In addition to the features shown in FIG. 1A, FIG. 1D illustrates first opening by showing first opening top part 8A(also shown in FIG. 1B) and first opening bottom part 8B, second opening by showing second opening top part 10A and second opening bottom part 10B (not shown here; instead shown in FIGS. 3A, 3B, and 4D), and third opening by showing third opening top part 12A and third opening bottom part 12B of the case. FIG. 1D also shows valve housing 32 and valve base 40.

The openings allow the fit of the valve ports in the closed state of the case as well as in the open states of the case. The first, second, and third openings have cross sections shaped large enough to accommodate the cross sections of the first port, second port, and the third port, respectively. The terms first, second, and third opening are defined in reference to the closed state of the case; however, as used herein they apply to the respective openings in all states of the case. For example, an opening that receives one of the ports, in some embodiments, is formed by two half openings, one of the half openings being a part of the top member, and the other half opening being a part of the bottom member. In some embodiments, some of the openings are a part of only the top or the bottom member or are composed of unequally shaped or sized parts of the top and bottom members.

In some embodiments, the housing of the valve includes the base of the valve, the first port, the second port, and the third port. Together with the handle and the operator, the housing constitutes the valve.

The valve base is the body of the valve housing excluding the ports. The base supports and makes up the areas around the ports.

Referring to FIG. 2A, a three-dimensional illustration of three-way valve case apparatus 70 is shown in an open state, whereas FIG. 2B shows three-way valve case apparatus 70 in a closed state.

For simplicity, only the terms open and closed are used here, the closed state referring to the states that substantially not directly allow the user to access the handle or change the state of the ports, and the open state referring to the states that enable the user to directly access the handle or change the state of the ports.

In alternative embodiments, the top and the bottom members of the case can be engaged to put the case into different positions. By the term “engaged,” it is referred to the act of moving the top member, the bottom member, or both top member and the bottom member relative to each other.

FIG. 3A illustrates a rendering of case 50. In addition to the previously introduced parts, shown are adhesive member 16, inner surface of the top member 18, inner surface of the bottom member 20, outer surface of the top member 22, and outer surface of the bottom member 24. Case 50 shown in FIG. 3A is in an open position, without a three-way valve being present. In contrast, the three-dimensional rendering of case 50 shown in FIG. 3B, also in an open position, has three-way valve 60 positioned inside it.

In certain embodiments, the bottom member of the case is adapted to be removably attached to a valve base. Such an adaptation can be accomplished by building a structure that fits a structural component of the three-way valve. The structure to be built into the bottom member of the case, in some embodiments can be large enough to fit around the valve or it can be smaller to fit into a part of the valve. In embodiments that have an inner surface of the bottom member that is adapted to receive the valve base, the attachment between the valve and the case can be created once, so that removal of the valve is not necessary, or it can be created in a way that allows the valve to be later removed and another one or a new one attached. In some embodiments, the case and the valve can be manufactured as a single piece. Alternatively, the manner of adapting the inner surface of the case, in some embodiments can include the use of adhesives, either directly between the case and the valve, or via the use of a separate member, such as a pad, that receives the adhesives on one or both sides.

In other embodiments, fasteners can be used to adapt a three-way valve to the inner surface of the bottom member. Examples include a snap, tie, securing means, strap, series of snaps, button, series of buttons, hook, series of hooks, elastic member, position adjuster, key-lock arrangement, lace, clip, series of clips, zipper, metal loop, roller loop, drawstring-and-cord lock arrangement, hook-to-hook arrangement, hook-and-loop arrangement, bungee cord-and-cord lock arrangement, block and tackle arrangement, rope and hook arrangement, chord and hook arrangement, snap hook, trap-lock, plastic trap-lock, magnet, slide, plastic slide, adhesive, as well as the likes, multiples, or combinations thereof. Fasteners known or developed in the future can be used so long as they allow opening and closing of the case.

The adhesive member, in embodiments that have it, enables the attachment of a three-way valve to the case. For example, the adhesive member, in some embodiments, enables attachment and removal of a third party three-way valve to the inner surface of the bottom member of the case. In alternative embodiments, the adhesive member is primarily designed for a single attachment; in others, for multiple attachment and removal cycles. The adhesive member, in some embodiments is a pad that has sticky properties. The pad or the adhesive member, in other embodiments, is permanently attached to the case; in others, it is supplied as a separate unit to be first attached to the case before the attachment of a valve. In alternative embodiments, the adhesive member is supplied as a non-adhesive member initially together with a substance or method of converting it to an adhesive member. An example for that would be a pad and glue, in which case a user can apply the glue to the pad to enable the attachment of a valve to the case. In some embodiments, the adhesive member is an integral part of the case, such as a portion of the inner surface of the bottom member that has adhesive properties toward a three-way valve. An embodiment for the adhesive member is a double sided tape pre-cut to the appropriate size and shape that is supplied with protective layers on both sides. A user can simply remove the protective layer from one side, apply the adhesive member to the inner surface of the bottom member, then remove the protective layer from the other surface, and affix the three-way valve. Multiple units of such an adhesive layer can be supplied, so that a user can conveniently use and dispose them, while keeping the strength of the layer equivalently strong and while being able to change valves as desired.

By the inner surface of the top or bottom member, it is referred to the surface of the respective member that is closer to the area where a valve base is to be placed than the remaining surface of such member, designated as the outer surface, is. This distinction is not defined by any of the surfaces being bounded by edges or by any of the surfaces being continuous, but only by the collective inner one being relatively closer to the valve base than the collective outer one.

The adjective smooth is used to refer to the property of the surface of the case that is substantially free of irregularities and projections. Some embodiments of the invention have a surface smoother than that of a three-way valve. Three-way valve 60 as seen in FIG. 3B has at least three projections, its valves, on its surface. In contrast, case 50 as seen in FIG. 3A has no substantial irregularities or projections on its surface when it is closed. Thus, the outer surface of the bottom member 24 is smooth, and the surface of the case 50 in general is smoother than the surface of the three-way valve 60. A more precise way of measuring smoothness of the surface of an object involves comparing the surface of an object with that of an ideal object with the same overall smooth shape, and calculating a least squares fit (e.g., non-linear least squares) between the two surfaces. Once the deviations from such a fit are obtained, sets of statistical estimates such as the mean, standard deviation, and the range of deviations can be calculated. The idealized shape to be used as the reference can be obtained from a mathematical equation describing the object, such as an ellipsoid, a superellipsoid, or a superquadric. The comparisons can also be made patch by patch, in which patches of the surface of the case are compared to flat or curved smooth idealized surfaces. The idealized surfaces can be similarly approximated by mathematical expressions, such as an expression for a plane in three-dimensions or a surface patch of an ellipsoid, superellipsoid, or superquadric. The size of the surface patch of the case can be determined by dividing the total surface area of the case by a number, such as 4, 6, 8, 10, 20, 40, 60, 80, 100, etc. In many cases, a qualitative visual inspection of the case can easily determine whether the case is smooth or not, without any recourse to mathematical approaches.

The adjective transparent is used to refer to the property of the case surface that allows the user to see through the case in a substantially clear way. For instance, the user can see a valve placed inside the case and determine the overall shape, position, and boundaries of the valve handle when looking from outside of a closed case. Some examples of materials that can be used to impart transparency to the case are polymethlamethacrylate, cellulose acetate butyrate, polycarbonate, and glycol modified polyethylene terephthalate. Transparent materials now known or developed in the future can be used so long as they allow for one to see through the case.

The adjective translucent is used to refer to the property of the case surface that allows the user to see through the case, but in a partial or diffuse way. For instance, a user looking through a translucent case surface would still be able to discern the overall shape of the valve handle and the position of the handle, but may find it more difficult, compared to looking through a transparent material, to make out the boundaries of the valve handle. Any of the transparent materials mentioned above, upon roughening, can become translucent instead of transparent. Some examples include translucent case acrylic, colored glass, and translucent ceramics. Translucent materials now known or developed in the future can be used so long as they allow for one to partially see through the case.

FIG. 4A shows case 50 in an open position, viewed from the front. The figure depicts top member 2, bottom member 4, hinge 6, closure members 14A-14D, and adhesive member 16. Closure members can be different from each other, for example, 14A and 14B can be similar to each other, and 14C and 14D can be similar to each other. In another embodiment, 14A can be similar to 14D, and 14B to 14C. In some embodiments, all four of the closure members are different from each other. As shown in FIG. 4A, closure member 14A interacts with 14C, and 14B interacts with 14D to enable closing the case. FIG. 4B shows case 50 from the side (left of FIG. 4A), with an additional feature, first opening depicted by showing first opening top part 8A and first opening bottom part 8B. FIG. 4C shows case 50 from another side (bottom of FIG. 4A), with the additional feature of third opening bottom part 12B shown. FIG. 4D is a perspective view of case 50, showing the additional features of second opening top part 10A, second opening bottom part 10B, inner surface of the top member 18, inner surface of the bottom member 20, outer surface of the top member 22, and outer surface of the bottom member 24.

In various embodiments, the case can be made from a plastic material. The plastics include acrylonitrile butadiene styrene, high impact polystyrene, high-density polyethylene, low-density polyethylene, melamine formaldehyde, phenolics, plastarch material, polyamides, polycarbonate, polycarbonate/acrylonitrile butadiene styrene, polyester, polyetheretherketone, polyetherimide, polyethylene, polyethylene terephthalate, polyethylene/acrylonitrile butadiene styrene, polylactic acid, polymethyl methacrylate, polypropylene, polystyrene, polytetrafluoroethylene, polyurethanes, polyvinyl chloride, polyvinylidene chloride, and urea-formaldehyde. Plastic or moldable materials now known or developed in the future can be used so long as they allow to mold the case, as described herein.

The specific plastic used, in some embodiments, can be a thermosetting polymer, such as polyester fibreglass systems, polyester resins, polyisocyanurate, polyurethanes, vulcanized rubber, bakelite, duroplast, urea-formaldehyde, melamine formaldehyde, epoxy, and polyimides.

The plastic used, in other embodiments, can also be a thermoplastic, such as acrylonitrile butadiene styrene, polyetheretherketone, polyamide, polyamide-imide, polyaryletherketone, polycarbonate, polyethylene terephthalate, polyimide, polymethyl methacrylate, polypropylene, polysulfone, polytetrafluoroethylene, polyvinyl chloride, high density polyethylene, low density polyethylene, self-reinforced polyphenylene, polyetherimide, celluloid, cellulose acetate, cyclic olefin copolymer, ethylene-vinyl acetate, ethylene vinyl alcohol, fluoropolymer, ionomers, KYDEX® acrylic/pvc alloy, liquid crystal polymer, polyacrylonitrile, polybutadiene, polybutylene, polycaprolactone, polychlorotrifluoroethylene, polyester, polyetherketoneketone, polyethersulfone, polysulfone, polyethylene terephthalate, polyethylene, polyhydroxyalkanoates, polyketone, polylactic acid, polymethylpentene, polyoxymethylene, polyphenylene oxide, polyphenylene sulfide, polyphthalamide, polystyrene, polytrimethylene terephthalate, polyurethane, polyvinyl acetate, polyvinylidene chloride, styrene-acrylonitrile, chlorinated polyethylene, and polycyclohexylene dimethylene terephthalate.

Methods used to manufacture various components of the apparatus (e.g., the case) include injection molding, compression molding, transfer molding, rotational molding, thermoforming, and additive manufacturing (e.g., 3D printing; stereolithography). Fewer or more pieces can be used to do the assembling in order to accomplish the same function, for example, two or three pieces can be molded into a single piece, or additional pieces can be added. Different materials that are now known or developed in the future can be substituted in place of others. The parts can have different sizes that the embodiments described herein, and the sizes and shapes of components can be changed and be different in different embodiments. For some embodiments, items can be pre-assembled; for other embodiments, they may need to be assembled by the users or by the medical practitioners.

The case can, in alternative embodiments, be made of an organic polymer, i.e., a compound with repeating units, which contains carbon atoms. Polymers can include synthetic, semi-synthetic, as well as natural organic compounds. Many of the plastics contain polymers or are derived from them. Apart from those mentioned as examples of plastics, polymers include gutta-percha, polyolefins, natural rubber, synthetic rubber, vulcanized rubber, latex, silicone, and cellulose.

Some of the methods of using the three-way valve case apparatus include performing acts such as closing the case, opening the case, moving or adjusting the handle of the three-way valve, connecting various kinds of tubing to any of the three ports, disconnecting various kinds of tubing from any of the three ports, attaching various capping structures to any of the ports of the three-way valve, aspirating fluids through the three-way valve, and flushing fluids through the three-way valve. Capping structures (e.g., valve caps, snap caps, snap-in caps, twisting caps, in addition to other structures capable of blocking the openings of the ports such as plugs) can be used to block and/or protect the port opening when no tubing is connected to the ports, and they can be made from materials similar to those that the case can be made from, such as various plastics. Methods of using the three-way valve case, in addition to including those that are used in conjunction with a three-way valve, include acts of placing a three-way valve into the case. A three-way valve can be placed into the case by various methods. For example, for cases having an adhesive member, a three-way valve can be positioned so that it adheres to this member and becomes stably situated within the case. Alternatively, a suitable surface area of a three-way valve (e.g., a surface area that is away from its handle) can be made adhesive by application of an adhesive and then positioned within the case. In another method, two-sided tape can be used for this same purpose: one side of the tape can be unprotected by removing its protecting layer or peeling it off from its roll, then it can be applied to the case or the valve, then the tape's second side can be unprotected by peeling off its protecting layer, and then the remaining component (e.g., case or the valve) can be affixed to the second side of the tape. Valves can also be placed by physically snapping them into place, in certain embodiments, to a complementary area of the case. In other embodiments, because of the snug dimensions of the case, simply placing a valve inside the case allows it to be used without a need for any elaborate fixation or the valve. The methods can be performed by the patient (as allowed/instructed by a health care professional) or by a practicing medical professional.

Among various embodiments of the present invention are kits that have cases as well as kits that have both cases and three-way valves. The cases and three-way valves included within kits can be any of those disclosed herein. In addition to cases and three-way valves, kits can include adhesive members (e.g., separately or as already part of at least one of the components) such as glue and double-sided tape; instruction manuals with tips, answers to common questions, lists of compatible alternative components (e.g., third party three-way valves), contact information for help, and cautionary remarks; duplicate parts; syringes for use with aspiration, flushing, or other acts; covers (e.g., padded, soft, cloth, etc.) for the case and/or other items that may be needed with such a three-way valve.

### Exemplification

The apparatus and the case were developed after extensive years of experience with three-way valves and their uses in medical settings. The initial recognition of the problem and the concept for solving it led to the creation of homemade casings, such as those made from gauze. Various initial attempts included enveloping the valves by wrapping them (e.g., with gauze) in addition to physically positioning them away from the patient's skin to a possible extent.

The case or the apparatus can be manufactured by injection molding or 3D printing. Industry standards that are in use or have been in use in the manufacturing of 3-way valves can be employed. The material can be a plastic (e.g., polypropylene) or any other material as long as it allows the case or the apparatus to function as it has been described in this document. Materials that are commonly used or usable for the manufacture of three-way valves can be similarly used for the case.

One embodiment of the case has a length, i.e., the distance between its first opening and third opening, of 1.75″, a width, i.e., the distance between its hinge and its second opening, of 1.625″, and a height, i.e., the distance between the furthest parts of its top and bottom members, of 1.125″.

The relevant teachings of all the references, patents, and patent applications cited herein are incorporated herein by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. An apparatus comprising:
a. a medical three-way valve comprising:
i. a housing having a first port, a second port, and a third port; and
ii. a valve operator having a mechanism, that when in use, allows for fluid communication between at least two ports; and
b. a case that receives the three-way valve, wherein the case is formed by a top member and a bottom member, the case comprising:
i. a hinge connecting the top member and the bottom member, wherein the hinge, when in use, allows the top member and the bottom member to be articulated between an open position and a closed position;
ii. a first opening, a second opening, and a third opening in the closed position to receive the first port, the second port, and the third port, respectively; and
iii. a closure member that can maintain the top member and the bottom member in the closed position;
wherein, when the case is in use and in the closed position, the valve operator is inaccessible to a user, and the three-way valve does not come into contact with the user's skin.

2. The apparatus of claim 1, wherein the closure member is a snap closure.

3. The apparatus of claim 1, wherein the hinge is a flexible hinge.

4. The apparatus of claim 1, wherein the first opening, the second opening, third opening, or a combination thereof is formed by portions of the top member, the bottom member, or both the top member and the bottom member.

5. The apparatus of claim 1, wherein the bottom member comprises a smooth outer surface.

6. The apparatus of claim 1, wherein the case has a surface smoother than a surface of the three-way valve.

7. The apparatus from claim 5, wherein the case is made from a plastic material.

8. The apparatus of claim 5, wherein the plastic material comprises a thermoplastic polymer, a thermosetting polymer, or a combination thereof

9. The apparatus of claim 8, wherein the thermoplastic polymer is selected from the group consisting of polyethylene, polypropylene, polystyrene, polyvinyl chloride, polytetrafluoroethylene, and a combination thereof.

10. The apparatus of claim 1, wherein the case is made from an organic polymer material.

11. The apparatus of claim 1, wherein the three-way valve further includes a handle in communication with the valve operator, and the case is adapted to cover the three-way valve and handle when in use.

12. The apparatus of claim 1, wherein at least a portion of the top member, the bottom member, or portions of both the top member and the bottom member are transparent or translucent.

13. A case adapted to cover a medical three-way valve, the three-way valve comprising a housing having a first port, a second port, a third port, and a valve base; a valve operator having a mechanism, that when in use, allows for fluid communication between at least two ports; and a handle that communicates with the valve operator; the case comprising:
a. a top member and a bottom member;
b. a hinge connecting the top member and the bottom member, wherein the hinge, when in use, allows top member and the bottom member to be articulated between an open position and a closed position; and
c. a first opening, a second opening, and a third opening in the closed position to receive the first port, the second port, and the third port, respectively; and
d. a closure member that can maintain the top member and the bottom member in the closed position;
wherein, when the case is in use and in the closed position, the valve operator and handle are inaccessible to a user, and the three-way valve does not come into contact with the user's skin.

14. The apparatus of claim 13, wherein the case has dimensions that prevent the three-way valve from sliding within the case.

15. The apparatus of claim 14, wherein the case, when in the closed position, has a width of between about 1.5″ and about 2.0″, and a length of between about 1.5″ and about 2.0″, and a height between about 0.75″ and about 1.2″.

16. The apparatus of claim 13, wherein the bottom member has an inner surface that is adapted to receive the valve base.

17. The apparatus of claim 16, further comprising an adhesive member that attaches the inner surface of the bottom member to the valve base.

18. The apparatus of claim 13, wherein the bottom member is adapted to be removably attached to the valve base.

19. A kit that comprises:
a. a three-way valve comprising:
i. a housing having a first port, a second port, and a third port;
ii. a valve operator having a mechanism, that when in use, allows for fluid communication between at least two ports; and
b. a case that receives the three-way valve, wherein the case is formed by a top member and a bottom member, the case comprises:
i. a hinge connecting the top member and the bottom member, wherein the hinge, when in use, allows the top member and the bottom member to be articulated between an open position and a closed position; and
ii. a first opening, a second opening, and a third opening in the closed position to receive the first port, the second port, and the third port, respectively;
iii. a closure member that can maintain the top member and the bottom member in the closed position;
wherein, when the case is in use and in the closed position, the valve operator is inaccessible to a user, and the three-way valve does not come into contact with the user's skin.

20. A method of using a case for a three-way valve, the three-way valve being attached to a catheter being used by an individual, the three-way valve comprising a housing having a first port, a second port, and a third port; a valve operator having a mechanism, that when in use, allows for fluid communication between at least two ports; wherein the case receives the three-way valve, wherein the case is formed by a top member and a bottom member, the case comprising a first opening, a second opening, and a third opening to receive the first port, the second port, and the third port, respectively; a hinge connecting the top member and the bottom member, wherein the hinge, when in use, allows the top member and the bottom member to be articulated between an open position and a closed position; and a closure member that can maintain the top member and the bottom member in the closed position; the method comprising the steps of:
a. placing the three-way valve in the case; and
b. engaging the top member and the bottom member so that the case is put into the closed position;
wherein the valve operator is inaccessible to the individual, and the three-way valve does not come into contact with the individual's skin.

21. The method of claim 20, further comprising the steps of:
a. engaging the top and bottom member so that the case is placed in the open position by a health care professional; and
b. engaging a handle that communicates with the valve operator as desired by a health care professional.

22. The method of claim 21, further comprising the steps of connecting tubing from the first port, the second port, the third port, or a combination thereof.

23. The method of claim 21, further comprising the steps of disconnecting tubing from the first port, the second port, the third port, or a combination thereof.

24. The method of claim 21, further comprising aspirating fluid through a port using the three-way valve.

25. The method of claim 21, further comprising flushing through a port using the three-way valve.
